(19) United States
(12) Patent Application Publication
(10) Pub. No.: US 2013/0172828 A1
(43) Pub. Date: Jul. 4, 2013

(54) ENDOSCOPIC GUIDE WIRE TRACK

(71) Applicants: Gary S. Kappel, Acton, MA (US); Paul Smith, Smithfield, RI (US); Larry Stanton, Burlington, MA (US); Ruth Cheng, Natick, MA (US); Kenneth R. Keene, Winchester, MA (US); Gerald R. Heller, Bedford, MA (US); Man Nguyen, West Roxbury, MA (US)

(72) Inventors: Gary S. Kappel, Acton, MA (US); Paul Smith, Smithfield, RI (US); Larry Stanton, Burlington, MA (US); Ruth Cheng, Natick, MA (US); Kenneth R. Keene, Winchester, MA (US); Gerald R. Heller, Bedford, MA (US); Man Nguyen, West Roxbury, MA (US)

(21) Appl. No.: 13/727,365

(22) Filed: Dec. 26, 2012

Related U.S. Application Data
(60) Provisional application No. 61/580,921, filed on Dec. 28, 2011.

(51) Int. Cl. A61B 17/00

(52) U.S. Cl. CPC A61B 17/00234
USPC 604/272; 606/1; 606/41; 606/167; 606/174; 606/205

(57) ABSTRACT

A medical device may include an end effector configured to perform a therapeutic procedure. The medical device may further include a guide having a modifiable shape. The guide may be adapted for insertion through a lumen of an access tube. Also, the end effector may be selectively attachable to the guide and configured to move along the guide.

### CROSS-REFERENCE TO RELATED APPLICATION

This application claims the benefit of priority from U.S. Provisional Application No. 61/580,921, filed Dec. 28, 2011, which is herein incorporated in its entirety.

### FIELD OF THE INVENTION

The present embodiments generally relate to medical devices, and, in particular, to medical devices providing a path for performance of medical procedures.

### BACKGROUND OF THE INVENTION

A transluminal device is a flexible instrument introduced into a patient's body for diagnostic or therapeutic purposes. Such a device is inserted into the body through a natural or an artificially created opening, and is delivered to a work site inside the body through a body channel, such as, for example, the esophagus, a blood vessel, etc. Examples of transluminal devices include endoscopes, guide tubes, catheters, etc. Although particular embodiments of the invention may be broadly applied to any transluminal device, for the sake of brevity and as an exemplary embodiment, the invention will be described as being applied to an endoscope in this disclosure.

Endoscopes are widely used for diagnostic and therapeutic purposes inside a body. There are many different uses for endoscopes, and typically, endoscope designs may be varied to optimize their performance for an intended application. For example, there are upper endoscopes for examination of the esophagus, stomach and duodenum, urethroscopes for examining the urethra and bladder, colonoscopes for examining the colon, angioscopes for examining the blood vessels and heart, bronchoscopes for examining the bronchi, laparoscopes for examining the peritoneal cavity, arthroscopes for examining joint spaces, etc. Each of these devices may include features to optimize their performance for the intended application.

In typical applications, a distal end of an endoscope is inserted into the body through a natural anatomic opening, such as, for example, the mouth, anus, vagina, etc. Endoscopes may also be inserted into the body through a surgically created incision. The distal end of the endoscope then proceeds from the point of insertion to a region of interest (work site) within the body by traversing a body channel. The endoscope may also include one or more channels configured to house various diagnostic or treatment devices. These diagnostic or treatment devices may include, among others, a light source, a viewing/imaging device, an irrigation channel, an aspiration channel, or the like. Therapeutic tools configured for specific therapeutic tasks (such as, for example, incision, grasping, stitching, etc.) may also be delivered to the work site through the channels of the endoscope. These and other devices that may be used with an endoscope are broadly referred to as therapeutic or diagnostic tools in this application.

In order to position a therapeutic tool for application of specific therapeutic tasks, an operator must typically manipulate controls at a proximal end of the medical device to bend, e.g., articulate, the distal end of the medical device to a particular orientation. Additionally, upon positioning of the endoscope to the appropriate orientation, an operator may also need to manipulate controls at the proximal end of the medical device to drive and/or actuate the therapeutic tool along a path within the body to perform a medical procedure. For example, an operator may need to direct a therapeutic tool around a tissue growth, such as a polyp, in order to treat and/or remove the polyp. Such manipulation requires the operator to possess an increased skill level. Further, the more a therapeutic tool must be directed, the more likely an error may occur and the longer a procedure may last. As such, there remains a need to provide a device for medical procedures which reduces the necessary skill level of the operator, and increase efficiency and patient safety.

### SUMMARY OF THE INVENTION

One embodiment of the invention is directed to a medical device. The medical device may include an end effector configured to perform a procedure. The medical device may further include a guide having a modifiable shape. The guide may also be adapted for insertion through a lumen of an access tube. Also, the end effector may be selectively attachable to the guide and configured to move along the guide.

In various embodiments, the medical device may include one or more of the following additional features: an access tube configured to receive the guide therein; wherein the guide includes a channel and the medical device further includes a tool disposed within the channel, wherein the tool is slidable within the channel and includes the end effector thereon; wherein the guide is configured to receive electric energy; a suction tube, wherein the guide is positioned along a distal end of the suction tube; wherein the guide further includes at least one securing mechanism configured to retain the shape of the guide; wherein the guide includes a first guide portion and a second guide portion, the first guide portion and second guide portion having a first unconnected configuration and a second connected configuration; wherein the end effector may include at least one of a retractable blade, scissors, a v-blade, a straight blade, a hooked blade, an injection needle, a grasping mechanism, and an energy probe; a tool having the end effector thereon, wherein the tool is configured to attach the end effector to the guide; wherein the tool includes a connector configured to attach the tool to the guide, the connector may include at least one of a channel, a hook, a clamp, and a magnet; wherein the connector includes a pair of flexible arms defining the channel, and wherein the channel is shaped to receive the guide therein; and wherein the channel may include one of a u-shaped channel and a c-shaped channel.

Another embodiment of the invention may be directed to a medical device. The medical device may include a tool having a distal end effector which may be configured to perform a procedure. The medical device may further include a guide which may be configured to transform from a collapsed configuration to an expanded configuration, wherein the expanded configuration defines a path. Also, the tool may be configured to be coupled with the guide and move relative to at least a portion of the path.

In various embodiments, the medical device may include one or more of the following additional features: wherein the tool includes a connector configured to couple the tool to the guide, the connector including at least one of a channel, a hook, a clamp, and a magnet; wherein the connector includes a pair of flexible arms defining the channel, wherein the channel is shaped to receive the guide therein; wherein the channel is at least one of a u-shaped channel and a c-shaped channel; wherein the tool includes at least one of a retractable blade, scissors, a v-blade, a straight blade, a hooked blade, an injection needle, a grasping mechanism, and an energy probe; an access tube configured to receive the guide therein; wherein the guide is configured to receive electric energy; and a suction tube, wherein the guide is positioned along a distal end of the suction tube.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of the specification, illustrate exemplary embodiments of the present disclosure and together with the description, serve to explain the principles of the invention.

FIG. 1 is a perspective view of a medical device having a track according to an embodiment of the present disclosure.

FIGS. 2A and 2B are perspective views of the medical device of FIG. 1, including embodiments of mechanisms for shaping the track.

FIGS. 3A and 3B are perspective views of the medical device of FIG. 1 having a track in a first configuration and a second configuration, respectively, including an alternative mechanism for shaping the track.

FIG. 4 is a perspective view of a medical device having a track according to another embodiment of the present disclosure.

FIG. 5A is a perspective view of a medical device having a track according to another embodiment of the present disclosure.

FIG. 5B is a perspective view of a medical device having a track according to another embodiment of the present disclosure.

FIG. 5C is a cross-sectional view of a portion of the track shown in FIG. 5B.

FIG. 6A is a perspective view of a track according to another embodiment of the present disclosure.

FIG. 6B is a cross-sectional view of FIG. 6A.

FIG. 7A is a perspective view of a medical device having a track according to another embodiment of the present disclosure.

FIG. 7B is a perspective view of a track according to another embodiment of the present disclosure.

FIG. 7C is a cross-sectional view of a track according to another embodiment of the present disclosure.

FIG. 7D is a perspective view of a track according to another embodiment of the present disclosure.

FIG. 8A is a side-view of a medical device having a track and including a suction cone according to another embodiment of the present disclosure.

FIG. 8B is a bottom-view of the cone of FIG. 8A.

FIGS. 9A-9C are perspective views of tracks including securing mechanisms.

FIG. 10 is a perspective view of a medical device have multiple tracks according to another embodiment of the present disclosure.

FIG. 11A is a perspective view of a medical device having a track according to another embodiment of the present disclosure.

FIG. 11B is a perspective view of a medical device having a track according to another embodiment of the present disclosure.

FIG. 12A is a perspective view of a medical device including an end effector tool and track guide according to another embodiment of the present disclosure.

FIGS. 12B and 12C are cross-sectional views of embodiments of the track guide according to FIG. 12A.

FIGS. 13A and 13B are perspective views of medical devices including exemplary connection mechanisms of embodiments of the present disclosure.

FIG. 14 is a perspective view of a medical device including an end effector tool according to another embodiment of the present disclosure.

FIGS. 15A-15I are perspective views of end effectors according to embodiments of the present disclosure.

### DESCRIPTION OF THE EMBODIMENTS

Reference will now be made in detail to embodiments of the present disclosure, examples of which are illustrated in the accompanying drawings. Wherever possible, the same reference numbers will be used throughout the drawings to refer to the same or like parts.

The terms “proximal” and “distal” are used herein to refer to the relative positions of the components of the exemplary medical device 10. When used herein, “proximal” refers to a position relatively closer to the exterior of the body of a patient or closer to the operator using medical device 10. In contrast, “distal” refers to a position relatively further away from the operator using medical device 10 or closer to the interior of the body of the patient.

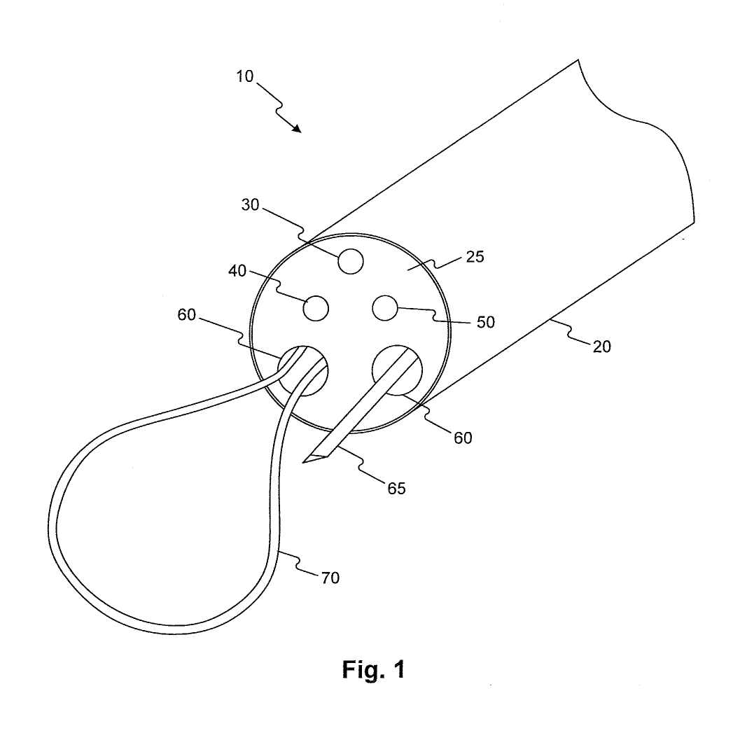

FIG. 1 depicts an exemplary medical device 10 that may be used for any therapeutic or diagnostic procedure, and the components thereof. The phrase “therapeutic or diagnostic procedure” is broadly used to indicate any medical procedure that may be performed by inserting a transluminal device, such as an endoscope, guide tube, catheter, or any other medical device, into the body through any anatomic opening. Medical device 10 may be used for performing surgery at a relative distance via medical instruments within device 10. The medical device 10 may be adapted for, but not limited to, trans-oral, trans-anal, trans-vaginal, trans-urethral, trans-nasal, trans-cranial, transluminal, laparoscopic, thorascopic, orthopedic, through the ear, and/or percutaneous access. The components of medical device 10 described below may be made of any suitable material capable of being inserted into the body, e.g., a suitable biocompatible material.

As shown in FIG. 1, medical device 10, may include a shaft 20 extending longitudinally from a proximal end to a distal end and terminating in a distal end face 25. Shaft 20 may include one or more channels 30, 40, 50, 60 each of which extends substantially longitudinally within shaft 20, and terminates in a port in distal end face 25. Channels 30, 40, 50, 60 may provide access to devices and facilities that may aid in performing a diagnostic or therapeutic task inside the body. In general, channels 30, 40, 50, 60 may be of any shape or geometry. In some embodiments, some or all of channels 30, 40, 50, 60 may be lined with a polymeric layer or another layer or coating to facilitate use. Channels 30, 40, 50, 60 may include one or more of, among others, an illumination/viewing/imaging channel 30, an aspiration channel 40, an irrigation channel 50, and one or more working channels 60.

Illumination/viewing/imaging channel 30 may include devices at the distal end configured to illuminate the work site. These devices may include, among others, bulbs, LED's, fiber optic cables and light guides. Illumination/viewing/imaging channel 30 may further include devices (such as a camera) at the distal end, configured to deliver an image of the work site external to the body of the patient. In some embodiments the camera may be a digital camera, such as a CCD or a CMOS camera. Illumination/viewing/imaging channel 30 may also include electric signal cables or wires that may run from the distal end of medical device 10 to the proximal end of medical device 10. Although depicted as a single channel in FIG. 1, the illumination/viewing/imaging channel 30 may, in an alternative embodiment, be divided amongst two (or more) channels including a first channel for illumination capabilities and a second channel for viewing/imaging capabilities. Additionally, any component of medical device 10 may further include a light transmitting material to deliver light.

Aspiration channel 40 may be configured to facilitate suction and/or fluid flow therethrough. As such, aspiration channel 40 may be in communication with a source of suction (i.e., vacuum) and/or fluid flow such as a pump at a proximal end of medical device 10. Irrigation channel 50 may be configured to facilitate fluid flow (including a vacuum) from the proximal end of medical device 10 to the distal end of medical device 10. In some embodiments, a proximal end of irrigation channel 50 may be attached to a source of fluid, and a distal end of irrigation channel 50 may include a nozzle to alter fluid flow. In some embodiments, fluid may flow from the proximal end of medical device 10 to the work site through irrigation channel 50. The fluid may then be removed from the work site, via suction, through aspiration channel 40. In some embodiments, aspiration channel 40 may also be configured to remove biological material along with fluid from the work site. For instance, a tissue sample along with fluid (delivered to the work site via irrigation channel 50) may be extracted out of the body of the patient through aspiration channel 40. In any event, both aspiration 40 and irrigation channel 50 may be used for infusion or aspiration of fluids and/or tissue.

Each of the working channels 60 may include a hollow cavity configured to deliver a tool 65, 210 to the work site. In general, working channel 60 may have any suitable shape, size, and configuration. In some embodiments, working channel 60 may have a substantially circular cross-section, while in other embodiments, the shape of the working lumen may correspond to the shape of tool 65 to be passed therethrough.

Additionally, working channel 60 may be configured to deliver a guide or track 70 to the work site. Track 70 may include an elongate piece of material extending from a proximal end of the medical device 10 towards the distal end of medical device 10 and bending back and extending toward the proximal end of medical device 10. Alternatively, track 70 may be configured as an elongate piece of material having a distal configuration, such as a loop. Track 70 may be delivered and/or moved to follow a single or compound path in any of three Cartesian directions, up/down, left/right, forward/backward. Track 70 may define a pre-determined, precise path for tool 65 to follow. As such, the path of tool 65 may be made increasingly accurate and safe. Further, use of track 70 may reduce the time necessary for a therapeutic procedure as the operator may simply guide tool 65 along path 70 rather than independently control tool 65 to move around or within the work site. Alternatively, as described below, track 70 may be provided with therapeutic procedure capabilities such that no additional tool 65 is required.

Track 70 may have a variety of configurations. For example, track 70 may have a modifiable shape and/or size in order to meet the needs of a particular procedure and/or patient. As such, track 70 may be formed of a semi-rigid or flexible material that may be shaped in a free-form manner or on a pre-determined template (not shown). The template may have any desired configuration. For example, the template may be configured to shape track 70 to dimensions corresponding to common polyps and/or tissue lesions such that the track may guide tool 65 around or near the polyp and/or lesion in order to treat a patient. Once positioned in the desired shape, the track may be treated, i.e. heated or chemically treated, such that track 70 may be made more rigid or has a shape memory effect. In one embodiment, track 70 may be manipulated by one or more moveable cores (not shown) configured to move inside of track 70. Alternatively, one or more moveable coaxial sheaths or tubes (not shown) may be moved along track 70. Either a cone or a sheath may be configured to follow the shape of the track 70 or may be configured to create a compound shape of track 70.

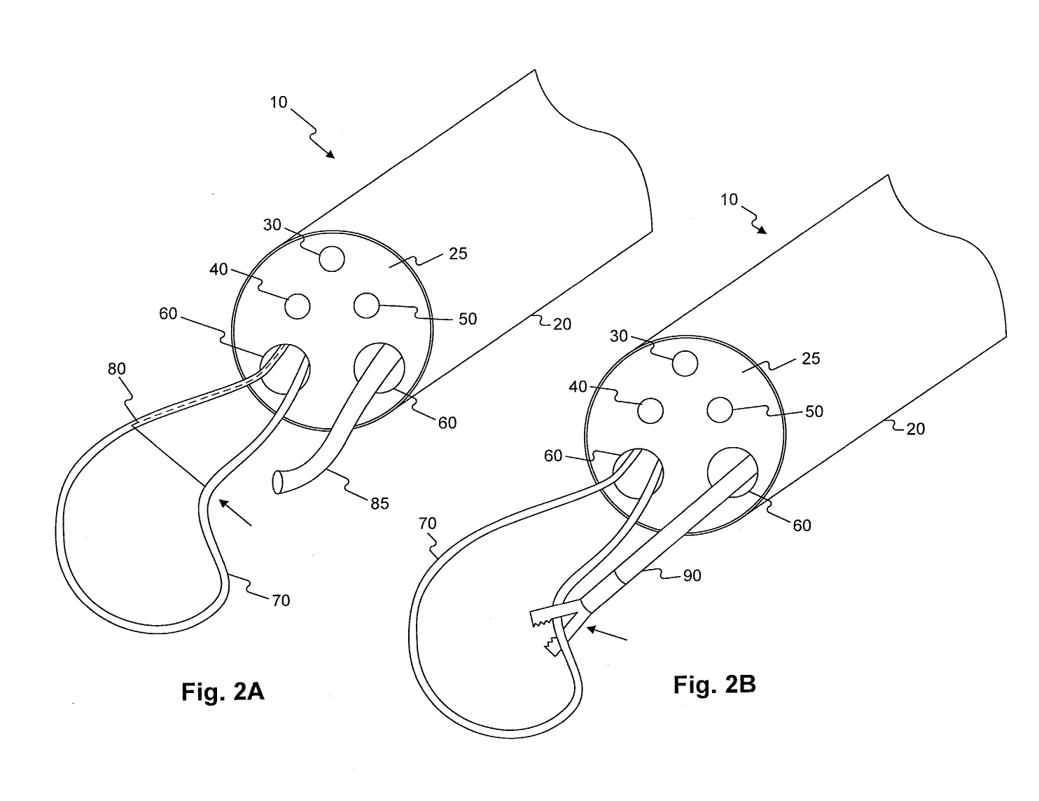

Alternatively, as shown in FIGS. 2A and 2B, track 70, being made of a modifiable material, may be manipulated via a pull-wire 80 and/or a manipulation tool 90 under direct visualization from a visualization scope 85. Visualization scope may be employed with any exemplary embodiments disclosed herein and may include a visualization mechanism, for example a camera, to transmit images to an operator of the track 70 while being shaped. Alternatively, a camera within illumination/viewing/imaging channel 30 can provide sufficient visualization.

As shown in FIG. 2A, pull-wire 80 may extend from a proximal end of medical device 10 through at least a first portion of track 70. Further, pull-wire 80 may exit a side wall of the first portion of track 70 and attach to a second portion of track 70, preferably opposite the first portion. In such a configuration, an operator may pull on a proximal end of pull-wire 80 such that the second portion of track 70 moves towards, i.e. is pulled, in the direction shown in FIG. 2A. Although not shown, medical device 10 may further include an oppositely arranged additional pull-wire 80 extending from a proximal end of medical device 10 through at least the second portion of track 70, exiting a side wall of the second portion of track 70, and attaching to the first portion of track 70. As such, the first portion of track 70 may move, i.e. is pulled, towards the second portion of track 70 in the direction opposite from that shown in FIG. 2A. As shown in FIG. 2B, the shape of track 70 may be modified via one or more manipulation tools 90. For example, manipulation tool 90 may be extended through a working lumen 60 and be configured to grasp and push track 70 in the direction shown in FIG. 2B or pull track 70 in a direction opposite to the direction shown in FIG. 2B.

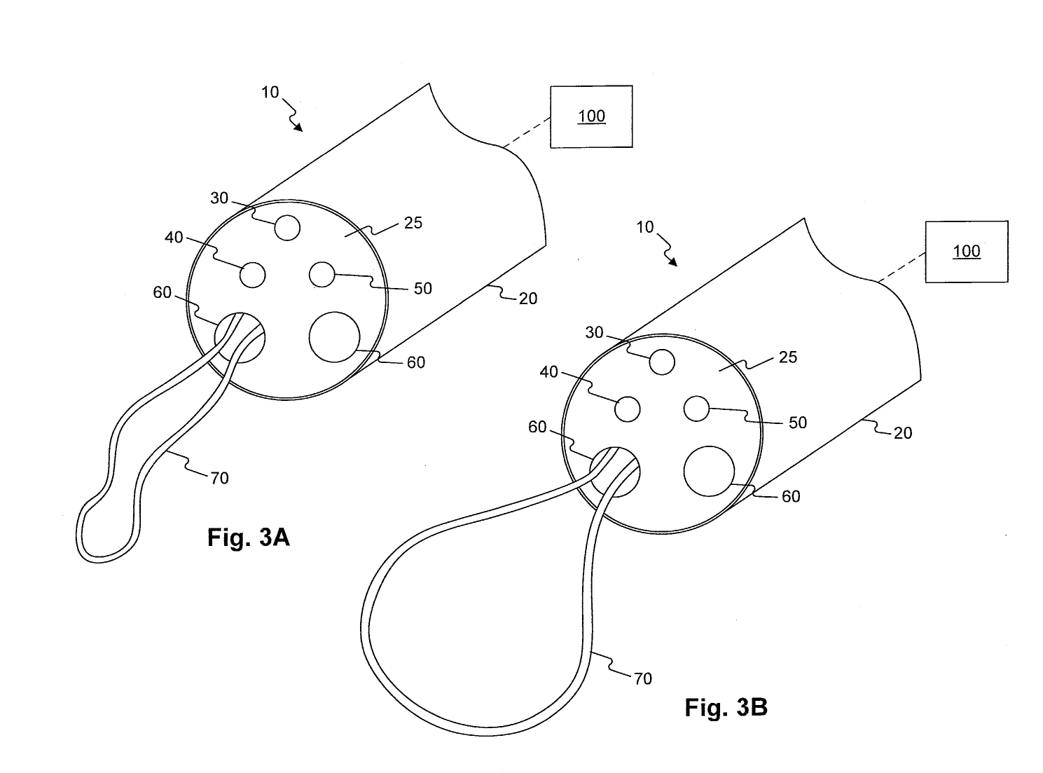

The shape of track 70 may also be modified through application of an excitation signal. In such an embodiment, track 70 may be made of a shape memory alloy (SMA) material. Non-limiting examples of SMA's that may be used to form track 70 include alloys of titanium-palladium-nickel, nickel-titanium-copper, gold-cadmium, iron-zinc-copper-aluminum, titanium-niobium-aluminum, iron-manganese-silicon, nickel-titanium, nickel-iron-zinc-aluminum, copper-aluminum-iron, titanium-niobium, etc. In some embodiments, track 70 may be made of nitinol. In such embodiments, track 70 may be subjected to an excitation signal originating from a signal generator 100. Signal generator 100 may be located at a proximal end of medical device 10 and electrically and/or thermally coupled to track 70. Signal generator 100 may be configured to deliver the excitation signal to track 70 thereby enabling track 70 to transform from a first configuration to second configuration by the application of heat or other stimuli. The first configuration may, for example, correspond to a contracted configuration (FIG. 3A), and the second configuration may, for example, correspond to an expanded configuration (FIG. 3B). In operation, track 70 may be delivered to the work site through working channel 60 in the first configuration as shown in FIG. 3A. After extension of track 70 through working channel 60, signal generator 100 may deliver the excitation signal to track 70 whereby track 70 may transform to the second configuration as shown in FIG. 3B.

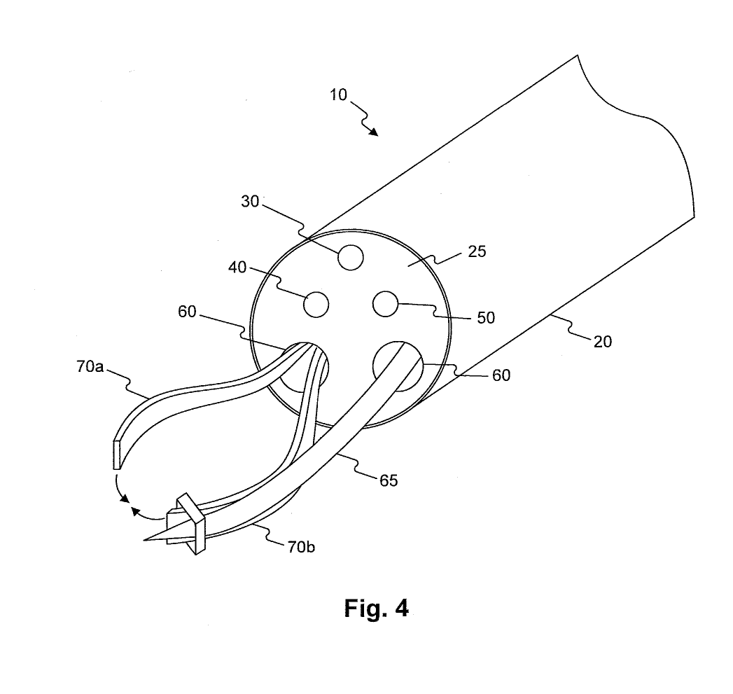

In an alternative embodiment, as shown in FIG. 4, track 70 may comprise two or more track portions 70a and 70b. Track portions 70a and 70b may extend alongside one another within working channel 60 and may be of any cross-sectional shape. For example, as shown in FIG. 4, track portions 70a and 70b may be configured as elongate members of generally rectangular cross-section. Division of track 70 into track portions 70a and 70b may prevent bunching or tangling of track 70. Additionally, such a configuration may allow tool 65 to attach more readily to track portions 70a and 70b. For example, after deployment of track portions 70a and 70b extending substantially longitudinally, tool 65 may slide onto a distal end of track portion 70b, via a clip or other suitable device. First and second track portions 70a and 70b may biased such that upon connection of tool 65, the first and second portions 70a and 70b close, i.e. move towards one another such that a single continuous track is formed.

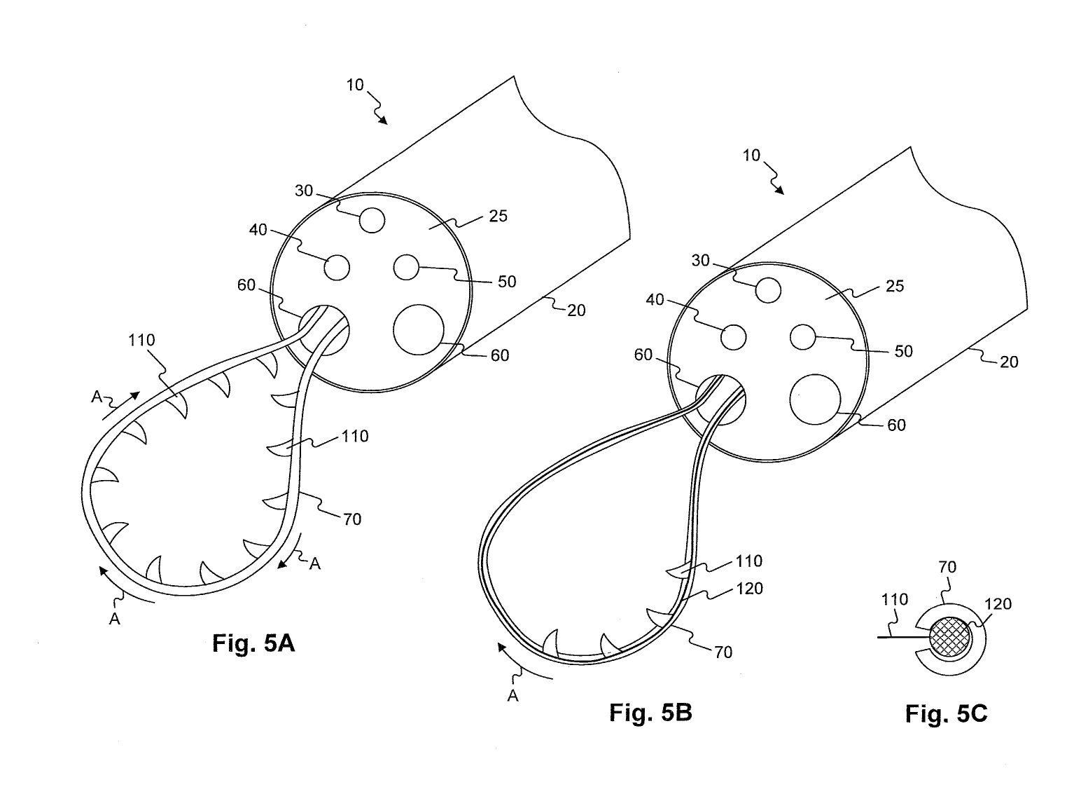

In an alternative embodiment, track 70 may include one or more end effectors 110. As shown in FIG. 5A, for example, each end effector 110 may include a blade, or any other structure capable of performing a therapeutic procedure. For example, end effectors 110 may include any of a variety of end effectors 220 discussed below. Further, track 70 may be configured to rotate either clockwise or counterclockwise. For example, track 70 may rotate in the direction A as shown in FIG. 5A. Alternatively, track 70 may rotate in a direction opposite of direction A, or capable of rotating in both directions. As track 70 rotates, one or more therapeutic procedures, such as cutting, may be performed.

In another exemplary embodiment, as shown in FIGS. 5B and 5C, track 70 may be non-rotary. In such a configuration, track 70 may be hollow and configured to receive an inner track 120 therein. Inner track 120 may be made rotatable/slidable within track 70. For example, track 70 may have a generally u-shaped or c-shaped cross-section and be configured to receive inner track 120. Track 70 may also include an opening or slot through which end one or more end effectors 110 extends. Inner track may further include one or more end effectors 110 thereon. Upon deployment of track 70 through working channel 60, inner track 120 with end effectors 110 may be rotated either clockwise or counterclockwise. As such, inner track 120 may rotate in the direction A as shown in FIG. 5B. Alternatively, inner track 120 may rotate in a direction opposite to the direction A. As inner track 120 rotates, one or more therapeutic procedures, such as cutting, may be performed.

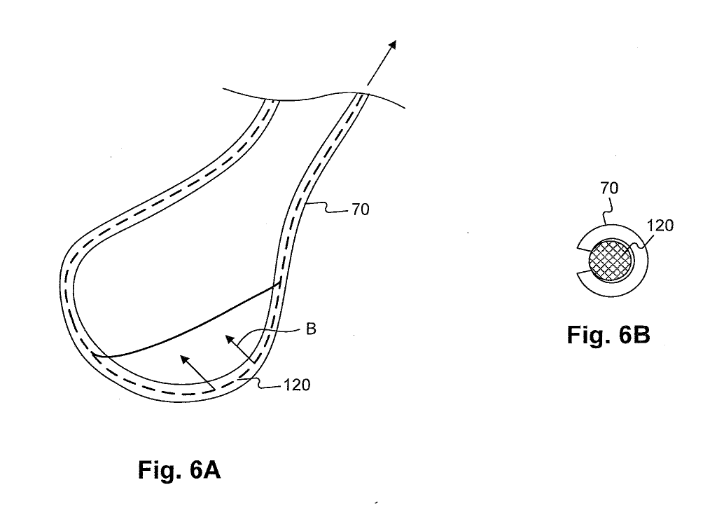

In an alternative exemplary embodiment, as shown in FIGS. 6A and 6B, track 70 may be hollow and configured to receive inner track 120 therein. Inner track 120 may be made moveable into and out of track 70 such that inner track 120 may contact tissue to perform a therapeutic procedure, such as cutting. For example, at least a portion of track 70 may have a generally u-shaped or c-shaped cross-section configured to receive inner track 120. The remaining portion of track 70 may fully encircle or enclose inner track 120. For example, a distal portion of track 70 may include the generally u-shaped or c-shaped cross-sectioned portion while the remainder of track 70 may have a circular cross-section. In such an embodiment, the portion of inner track 120 within the distal portion of track 70 may be made moveable into and out of track 70 such that inner track 120 may contact tissue. The remainder of the track 70, having a circular cross-section, may prevent the portion of the inner track 120 received therein from moving into and out of track 70. Inner track 120 may be configured to exit track 70 in a direction B upon being tensioned at a proximal end. As such, inner track 120 may perform a therapeutic procedure only a preset distance from track 70. This may allow for increased control and versatility in performing a therapeutic procedure. For example, an operator may perform a therapeutic procedure on one side of the work site at a time.

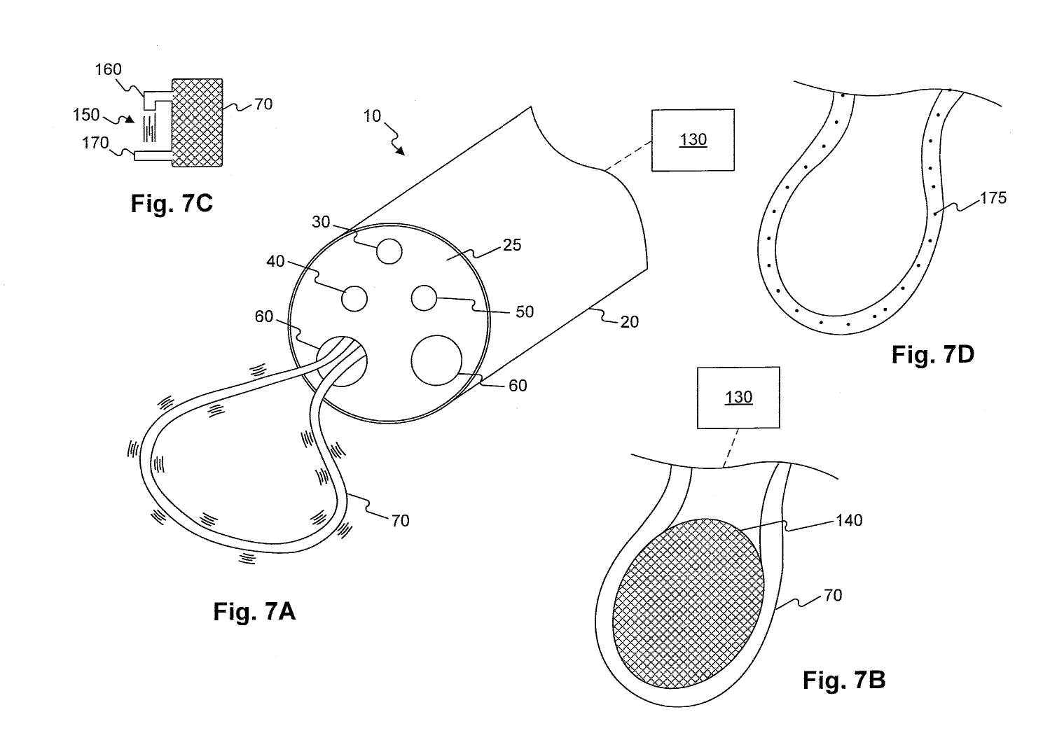

In another embodiment, as shown in FIG. 7A, track 70 may be electrically coupled to a source 130. Source 130 may be configured to deliver an electric current signal to track 70. For example track 70 may coagulate, cauterize, dissect, burn, and/or cut tissue at the work site upon being energized by the electric current signal from source 130. Alternatively, as shown in FIG. 7B, track 70 may be configured to deploy an radio-frequency (RF) conducting mesh 140. Mesh 140 may be configured to receive an RF signal from source 130 and ablate tissue at the work site. Mesh 140 may be in the shape of an oval as shown in FIG. 7B. Alternatively, mesh 140 may be in any appropriate shape to ablate tissue at the work site, such as, for example, a square, a rectangle, or any irregular shape.

As shown in FIG. 7C, track 70 may include a fluid or laser jet device 150. Jet device 150 may extend from a side portion of track 70 and may include a port 160 configured to direct fluid (e.g., saline or water) or laser energy towards platen 170. Although shown as extending to the left of a center of track 70, platen 170 and port 160 are not so limited. Rather, platen 170 and port 160 may extend in any direction from track 70 (e.g., towards and inside or outside of track 70, or above or below track 70) so as to achieve a desired therapeutic procedure. In operation, tissue received between port 160 and platen 170 may be treated. For example, jet device 150 can cause laser energy or high pressure fluid from port 160 towards platen 170 to cut tissue received therebetween. In such embodiments, port 160 may be in communication with a source of laser energy or fluid at the proximal end of medical device 10.

As shown in FIG. 7D, track 70 may include one or more needle projections and/or ports 175. The needle projections and/or ports 175 may disposed along track 70 and may be configured to simultaneously, or individually, inject fluid, such as saline, solution and/or water to the work site. In some embodiments, the fluid injected through projections and/or ports 175 may be pressurized such that track 70 is provided with fluid jet capability. Although projections and/or ports 175 are shown as disposed as facing above track 70, other orientations are possible. For example, projections and/or ports 175 may face upwards, downwards, radially inwards, radially outwards, or some combination thereof without departing from the scope of the disclosure. As such, orientation of the projections and/or ports may be selected to perform a desired therapeutic procedure. Additionally, projections and/or ports 175 may be configured to deliver suction, i.e. vacuum, as needed. The application of suction through projections and/or ports 175 may aid to retain track 70 in place while a procedure is performed.

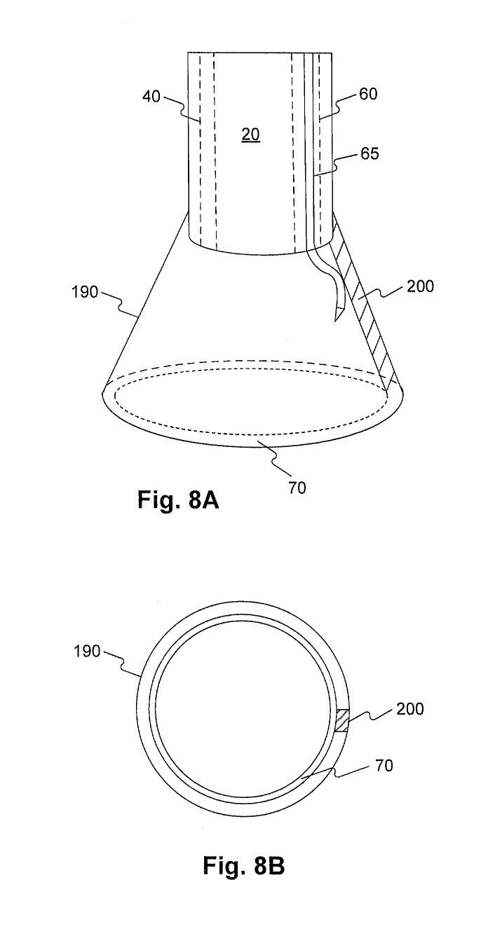

In another exemplary embodiment, as shown in FIGS. 8A and 8B, the distal end of shaft 20 may be associated with a suction cone 190. Suction cone 190 may extend distally of shaft 20 and have any shape required to achieve a desired therapeutic procedure. For example, as shown in FIGS. 8A and 8B, suction cone 190 may have a circular cross-section. Alternatively, the cross-sectional shape of suction cone 190 may be selected to correspond with the dimensions of common polyps and/or tissue lesions. Suction cone 190 may surround the polyp and/or lesion in order to treat a patient. Suction cone 190 may be used to apply suction to the work site. For example, suction cone 190, in conjunction with aspiration channel 40, may apply suction via a source of suction such as a pump at a proximal end of medical device 10.

As shown in FIG. 8A, a side wall of suction cone 190 may include a channel 200 and a distal rim of suction cone 190 may include track 70. That is, track 70 may be positioned along the inner wall and/or a distal end of suction cone 190. Alternatively, track 70 may be positioned along a portion or a perimeter of any portion of suction cone 190. As such, track 70 may extend along and between any plane passing through suction cone 190. Additionally, track 70 may be configured in any shape, including a spiral shape, along an inside wall of suction cone 190. In such a configuration, tool 65 may be guided through working channel 60 of medical device 10, along channel 200 in suction cone 190, to track 70. Although shown as inside cone 190, channel 200 may alternatively be provided along an outside wall of suction cone 190. Use of suction through suction cone 190 may aid in retaining track 70 in place. For example, upon insertion into the body of a patient, suction cone 190 may be placed over the work site. After placement, suction may be applied through aspiration channel 40. As such, suction cone 190 may be temporarily fixed in place on tissue at the work site. Such an engagement prevents accidental displacement of track during use, thereby improving accuracy of an operator and reducing potential damage to healthy tissue adjacent to the work site.

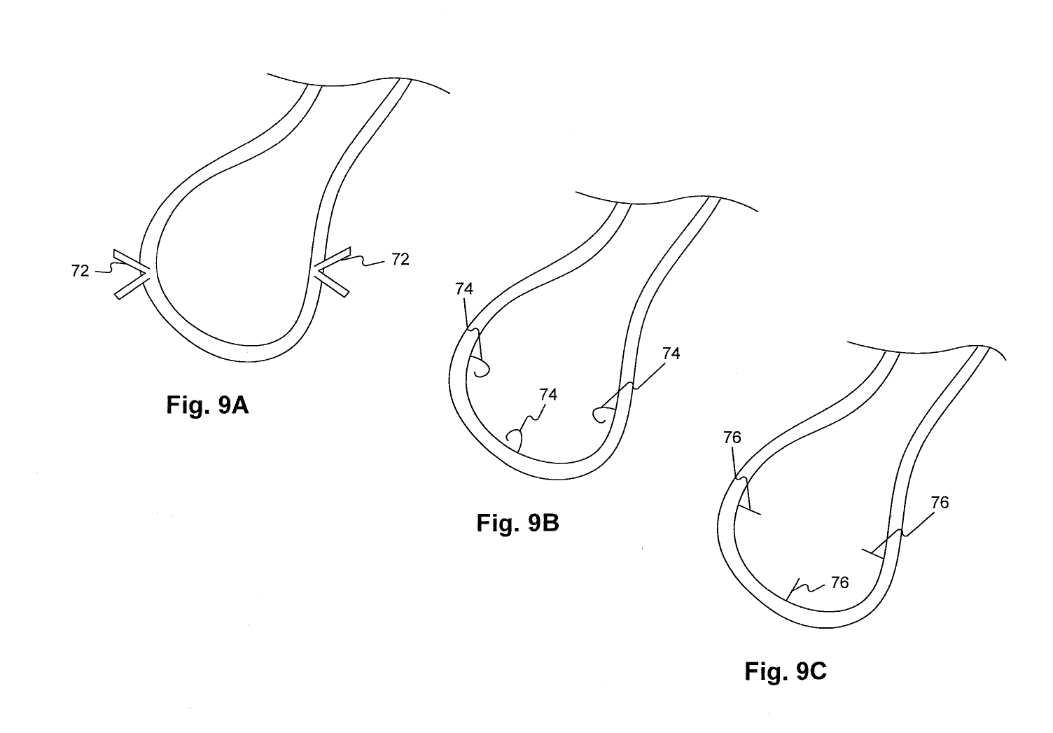

As shown in FIGS. 9A-9C, track 70 may include one or more anchoring mechanisms configured to engage tissue and retain track 70 in a particular shape and/or location within the body of the patient. For example, anchoring mechanisms, may include clips 72 (FIG. 8A), hooks 74 (FIG. 8B), and/or barbs 76 (FIG. 8C). Moreover, the anchoring mechanisms may be made retractable. As such, track 70 may be extended through working channel 60 while clips 72, hooks 74, and/or barbs 76 are in a retracted positioned. Upon attainment of the desired track 70 shape, clips 72, hooks 74, and/or barbs 76 may be actuated, manually or automatically, such that clips 72, hooks 74, and/or barbs 76 engage tissue and secure track 70 in place. Additionally, an operator may employ clips 72, hooks 74, and/or barbs 76 to mark and/or score the work site. Further, one track 70 has been anchored in a desired position, track 70 may be expanded and/or manipulated to stretch and or make tissue taut such that tissue may be dissected with greater ease and accuracy. Further, the track 70, including one or more anchoring mechanisms, may be used to retract tissue. That is, track 70 may be positioned such that upon expansion of track 70, track 70 and the one or more anchoring mechanisms may manipulate tissue so as to retract tissue away from the work site.

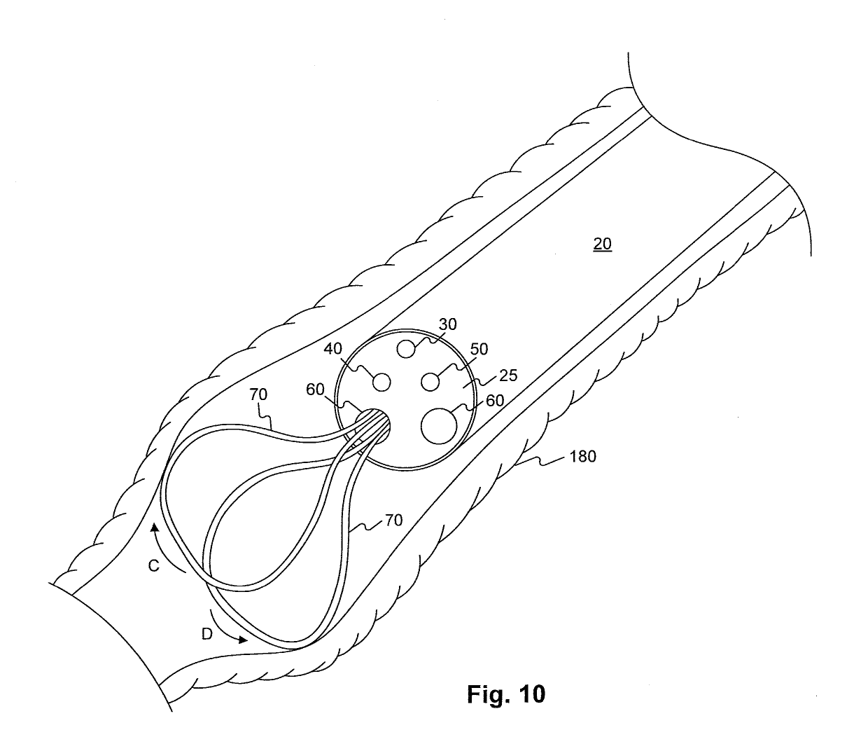

As shown in FIG. 10, multiple tracks 70 may be deployed through shaft 20. For example, two tracks 70 may be extended through the same working channel 60. Alternatively, each track 70 may be extended through separate working channels 60 (not shown). Tracks 70 may be manipulated within a body lumen 180 of a patient so as to create an enlarged space therein. That is, upon deployment of the medical device 10 into body lumen 180, tracks 70 may be extended through working channel(s) 60 of medical device 10. After deployment, tracks 70 may be separated, i.e. moved away from one another so as to increase a volume of body lumen 180. For example, movement of a first track 70 in the direction C and movement of a second track 70 in the direction D may place tension on body lumen 180. Under such tensions, an inner circumferential wall of body lumen 180 may spread so as to increase in diameter. The tracks may be separated by moving in opposite directions from one another, such as up/down or left/right. Alternatively, tracks 70 may be rotated or tilted away from one another.

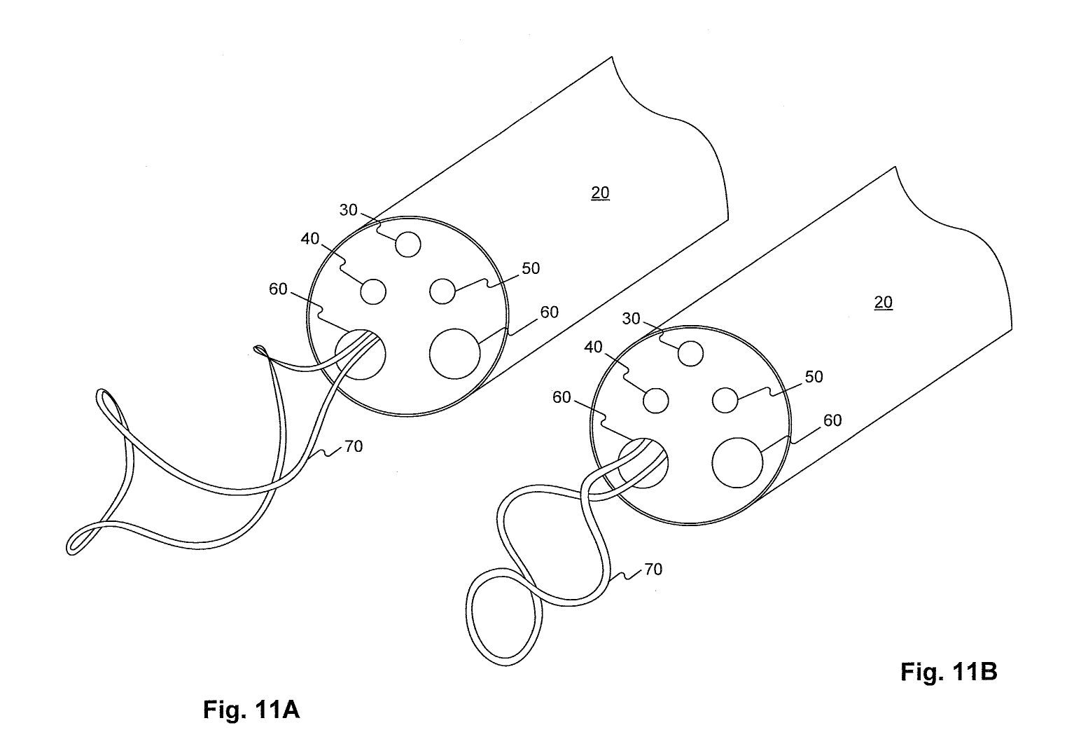

Although generally depicted as arcuate in shape, track 70 may employ any configuration useful for a therapeutic procedure. For example, as shown in FIGS. 11A and 11B, track 70 may include any spiraling shape such that a tool 65 may travel around a 360° path.

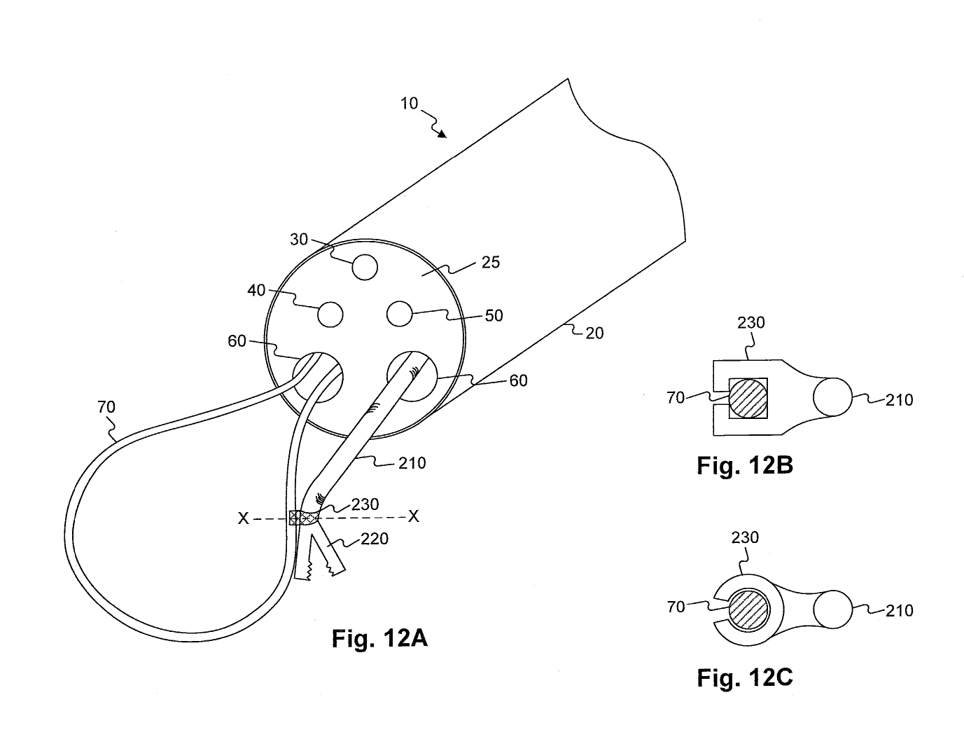

As shown in FIG. 12A, a tool 210 which may be sized and configured for insertion through working channel 60 of shaft 20. Tool 210 may be used to carry a manipulator for shaping track 70 as shown in FIG. 2B and described above. Alternatively, tool 210 may include an end effector 220 for performing a therapeutic procedure. Tool 210 may include a connector 230 configured to connect tool 210 to track 70. Connector 230 may be configured as a cuff having a u-shaped (FIG. 12B) or c-shaped (FIG. 12C) cross-section. Connector 230 may extends from tool 210 and surrounds track 70. Although depicted as being open on the left side of connector 230, other configurations may be employed. For example, connector 230 may be open to the top, the bottom, or the right side such that tool 210 and end effector 220 may be guided on bottom, top, or inside of track 70, respectively. Connector 230 may include a sufficiently flexible material and/or a living hinge configured to deflect outwardly upon engagement with track 70, where the material is also sufficiently rigid so as to retain track 70 therein. That is, as connector 230 is directed into engagement with track 70, connector 230 may expand so as to receive track 70 therein. Further, upon insertion of track 70 into connector 230, track 70 may be held within connector 230. Upon connection of connector 230 to track 70, an operator may guide, i.e. push or pull, tool 210 along track 70 such that end effector 220 performs a therapeutic procedure at the work site. The angle at which end effector 220 is positioned with respect to track 70 may be a function of how tool 210 is guided. That is, in an embodiment where tool 210 is pushed along track 70, the angle end effector 220 is disposed with respect to track 70 may be larger. On the other hand, in an embodiment where tool 210 pulled along track 70, the angle end effector 220 is disposed with respect to track 70 may be smaller.

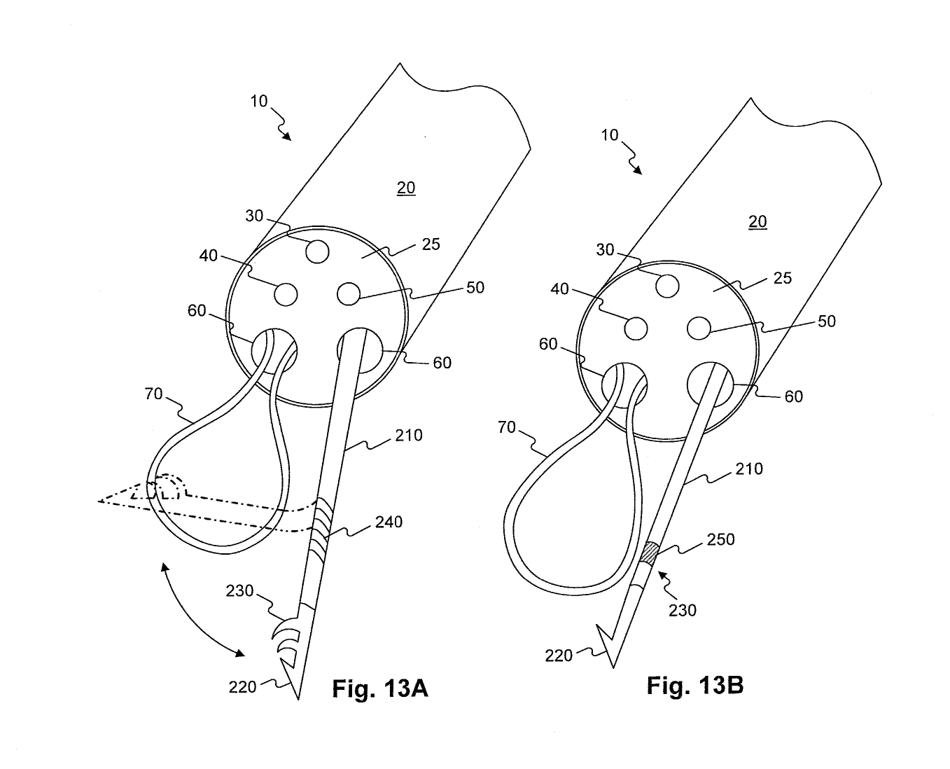

Alternative connections between tool 210 and track 70 are possible. For example, tool 210 may be keyed to track 70 or track 70 may be keyed to tool 210. As shown in FIG. 13A, a hook-shaped connector 230 may be employed. Alternatively, connector 230 may include a magnet 250 as shown in FIG. 13B. In such an embodiment, track 70 may be made of a magnetic material such that upon deployment of tool 210 through working channel 60, magnetic interaction between track 70 and magnet 250 acts to connect tool 210 to track 70. Further, track 70 may be increased in size so as to create a large surface area for magnetic interaction with magnet 250.

Regardless of the configuration of connector 230, tool 210 may include an articulation joint 240 to facilitate connection. For example, as shown in FIG. 13A, articulation joint 240 may include a series of rings that aid in bending a distal end of tool 210 in a desired direction, upon actuation by a controller at the proximal end of medical device 10. Alternatively, articulation joint 240 may include a portion having different material properties, such as increased flexibility.

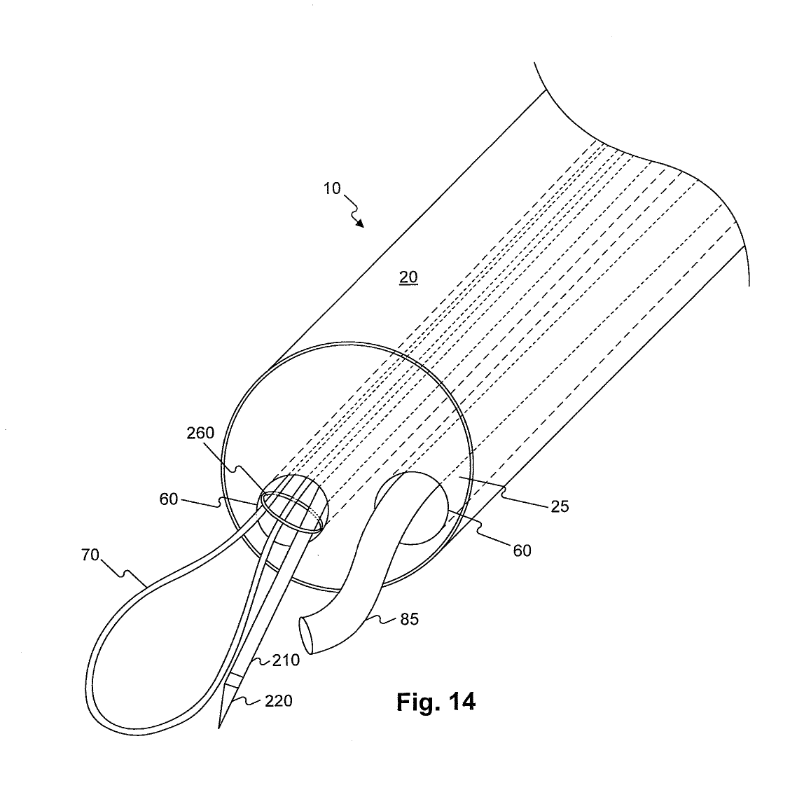

Although shown as extending through a separate working channel 60 than track 70 in FIGS. 12A, 13A, and 13B, in an alternate embodiment tool 210 and track 70 may extend through a common working channel 60. For example, as shown in FIG. 14, tool 210 and track 70 are deployed through a common working channel 60. Such an embodiment may ease connection of tool 210 to track 70. For example, where tool 210 and track 70 are deployed through a common lumen, tool 210 may be connected to track 70 at a proximal end of medical device prior to insertion of either track 70 or tool 210 in common working channel 60. Alternatively, after insertion of track 70 into common working channel 60, tool 210 may be connected to a proximal end of track 70 and then subsequently deployed through common working channel 60. In such a configuration, medical device 10 may further include a ring 260. Ring 260 may be independent and movable with respect to each of track 70 and tool 210. Alternatively, ring 260 may be formed integral with either one of track 70 and tool 210. Ring 260 may be employed to maintain tool 210 in close proximity with track 70. Further, ring 260 may prevent tangling of track 70 and tool 210 within common working channel 60. As described above, working channel 60 may include any conceivable geometry. For example, working channel 60 in FIG. 14 may be elliptical or oblong in shape to accommodate track 70 and tool 210.

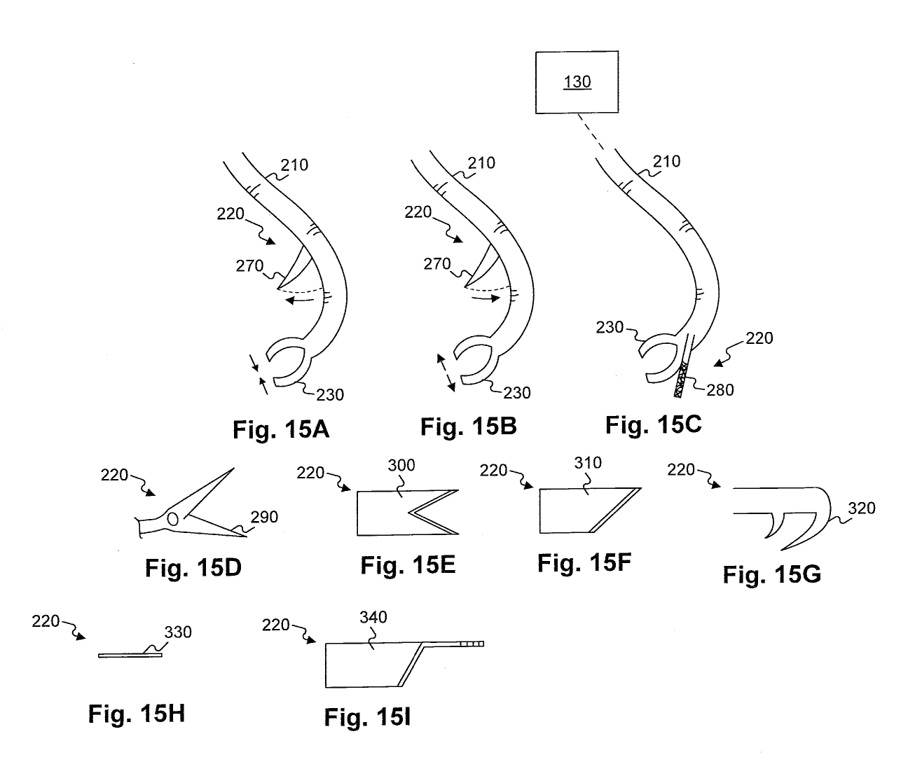

In another embodiment, as shown in FIGS. 15A and 15B, tool 210 may include a retractable end effector, such as retractable blade 270. Retractable blade 270 may move between a first unextended position and a second deployed position. For example, while in the first unextended position, retractable blade 270 may lie substantially flat along and/or within tool 210. Upon actuation, retractable blade 270 may move towards the second deployed position in which retractable blade 270 moves outward from tool 210. While in the second deployed position, retractable blade 270 may perform a therapeutic procedure. Retractable blade 270 may be actuated manually through controls and/or a pull wire (not shown) extending through tool 210 and connected to a controller at the proximal end of the medical device 10.

In an embodiment including retractable end effector such as retractable blade 270, connector 230 may be adjustable. That is, connector 230 may be actuated to open and close depending on the position of retractable blade 270. Connector 230 may be actuated manually through controls and/or a pull wire (not shown) extending through tool 210 and connected at the proximal end of medical device 10. Alternatively, connector 230 may be actuated automatically upon actuation of retractable blade 270. For example, connector 230 may be configured to close when retractable blade moves towards or is in the deployed position (FIG. 15A). As such, connector 230 may clamp around track 70 to secure tool 210 and retractable blade 270 to track 70. As retractable blade 270 moves toward or is in the unextended position, connector 230 may open such that tool 210 and retractable blade 270 may be disconnected from track 70 (FIG. 15B).

In another embodiment, as shown in FIG. 15C, a proximal end of tool 210 may be connected to source 130. Source 130, as described above, may be configured to deliver an electric current signal to tool 210 and/or end effector 220. End effector 220 may include a probe 280 configured to receive the electric current signal supplied by source 130. For example, probe 280 may coagulate, cauterize, dissect, burn, and/or cut tissue at the work site upon being energized by the electric current signal from source 130.

End effector 220 may include any type of end effector 220 capable of performing a desired therapeutic procedure. For example, end effector 220 may include scissors 290 (FIG. 15D). Scissors 290 may be of any shape and/or size configured to achieve a desired therapeutic effect. Further, scissors 290 may be bipolar or monopolar. Alternatively, end effector 220 may include any of a variety of blades such as a v-blade 300 (FIG. 15E), a straight blade 310 (FIG. 15F), and/or a hooked blade 320 (FIG. 15G). Blades 300, 310, and 320, similarly to scissors 290, may be of any shape and/or size configured to achieve a desired therapeutic effect, and may be configured to receive energy for electrocautery or coagulation. Additionally, end effector 220 may include an injection needle 330 (FIG. 15H). Needle 330 may be in communication with a fluid source to provide, for example, saline, solution, water, and or medicines, located at a proximal end of medical device 10, and may be configured to inject such fluid as needed to perform a therapeutic procedure.

Further, end effector 220 may include multiple devices for performing a therapeutic procedure. For example, as shown in FIG. 15I, end effector 220 may include a combination blade and probe 340. In such an embodiment, combination blade and probe 340 may be employed such that the probe may separate tissue at the work site and the blade may follow to perform a therapeutic procedure, such as cutting tissue.

In further embodiments, track 70 may provide power to tool 210, tool 210 may provide power to the track 70, and/or the connector 230 may complete a monopolar circuit. Track 70 and tool 210 may comprise opposite charges thus creating a bipolar circuit. Connector 230 may be configured to insulate track 70 and tool 210 from each other.

Medical device 10 may employ any variety of combinations of embodiments disclosed herein in order to achieve a therapeutic effect. Indeed, any of the embodiments disclosed herein may be used in conjunction with any other. For example, medical device 10 may include a tool 210 connected via connector 230 to a track 70 wherein each of the tool 210 and track 70 may be provided with therapeutic capabilities. By way of example only, track 70 may include tissue effector mechanism(s) 110 (FIGS. 5A-C), inner track 120 (FIGS. 6A-B), an electrically energized track (FIGS. 7A and 7B), a water and/or laser jet 150 (FIG. 7D), a plurality of injection ports 175 (FIG. 7D), and/or combinations thereof. Further, tool 210 may also include a retractable end effector such as retractable blade 270 (FIGS. 15A and 15B), probe 280 (FIG. 15C), scissors 290 (FIG. 15D), v-blade 300 (FIG. 15E), straight blade 310 (FIG. 15F), hook blade 320 (FIG. 15G), needle 330 (FIG. 15H), and/or combinations thereof. As such, any combination of embodiments disclosed herein may be selected to achieve a desired therapeutic effect.

As will be appreciated by one of ordinary skill in the art, the presently disclosed injection embodiments may have numerous advantages. For example, the disclosed track 70 may define a pre-determined, precise path for a tool 65, 210 to follow. As such, the path of the tool 65, 210 may be made increasingly accurate and safe. Further, use of track 70 may reduce the time and skill necessary for a therapeutic procedure as the operator may simply guide the tool 65, 210 along path 70 rather than independently control tool 65, 210 to move around or within the work site. Alternatively, track 70 itself may be provided with therapeutic procedure capabilities such that no additional tool 65, 210 is required. Also, track 70 may have a modifiable shape and/or size in order to meet the needs of a particular patient. As such, track 70 may employ an unlimited number of track designs and shapes and may be adapted to treat any required work site.

As noted above, any aspect set forth in any embodiment may be used with any other embodiment set forth herein. For example, in any embodiment, multiple tools may follow one track 70. The tools may interact to perform a procedure. In some embodiments, one tool may grasp and interact and/or apply tension to tissue while another tool may cut or dissect tissue. In embodiments employing multiple tools, some tool may be configured to interact with track 70 while tools may not. Every device and apparatus set forth herein may be used in any suitable medical procedure, may be advanced through any suitable body lumen and body cavity, and may be used for treatment of any suitable body portion. For example, the apparatuses and methods described herein may be used in any natural body lumen or tract, including those accessed orally, vaginally, or rectally.

The many features and advantages of the present disclosure are apparent from the detailed specification, and thus, it is intended by the appended claims to cover all such features and advantages of the present disclosure which fall within the true spirit and scope of the present disclosure. Further, since numerous modifications and variations will readily occur to those skilled in the art, it is not desired to limit the present disclosure to the exact construction and operation illustrated and described, and accordingly, all suitable modifications and equivalents may be resorted to, falling within the scope of the present disclosure. For example, in any embodiment, track 70 may include a removable sheath (not shown) surrounding track 70. The sheath may be removed to reveal track 70 and or one or more anchors. Additionally, in embodiments including a plurality of tracks 70, each track 70 may be configured to cooperate with a separate tool 210, or may be designed to allow at least one tool 210 to be moved from any one track 70 to another.

What is claimed is:

1. A medical device, comprising:
an end effector configured to perform a procedure; and
a guide having a modifiable shape and adapted for insertion through a lumen of an access tube;
wherein the end effector is selectively attachable to the guide and configured to move along the guide.

2. The medical device of claim 1, further comprising:
an access tube configured to receive the guide therein.

3. The medical device of claim 1, wherein the guide includes a channel, the medical device further comprising:
a tool disposed within the channel, wherein the tool is moveable relative to and within the channel and includes the end effector thereon.

4. The medical device of claim 1, wherein the guide is configured to receive electric energy.

5. The medical device of claim 1, further comprising:
a plurality of tools, each of the plurality of tools configured to move relative to the guide.

6. The medical device of claim 1, wherein the guide further includes at least one securing mechanism configured to retain the shape of the guide.

7. The medical device of claim 1, wherein the guide includes a first guide portion and a second guide portion, the first guide portion and second guide portion having a first unconnected configuration and a second connected configuration.

8. The medical device of claim 1, wherein the end effector includes at least one of a retractable blade, scissors, a v-blade, a straight blade, a hooked blade, an injection needle, a grasping mechanism, and an energy probe.

9. The medical device of claim 1, further comprising:
a tool having the end effector thereon, wherein the tool is configured to attach the end effector to the guide.

10. The medical device of claim 9, wherein the tool includes a connector configured to attach the tool to the guide, the connector including at least one of a channel, a hook, a clamp, and a magnet.

11. The medical device of claim 10, wherein the connector includes a pair of flexible arms defining the channel, wherein the channel is shaped to receive the guide therein.

12. The medical device of claim 11, wherein the channel is at least one of a u-shaped channel and a c-shaped channel.

13. A medical device, comprising:
a tool having a distal end effector configured to perform a procedure;
a guide configured to transform from a collapsed configuration to an expanded configuration, wherein the expanded configuration defines a path; and
wherein the tool is configured to be coupled with the guide and move along and relative to at least a portion of the path.

14. The medical device of claim 13, wherein the tool includes a connector configured to couple the tool to the guide, the connector including at least one of a channel, a hook, a clamp, and a magnet.

15. The medical device of claim 14, wherein the connector includes a pair of flexible arms defining the channel, wherein the channel is shaped to receive the guide therein.

16. The medical device of claim 15, wherein the channel is at least one of a u-shaped channel and a c-shaped channel.

17. The medical device of claim 13, wherein the tool includes at least one of a retractable blade, scissors, a v-blade, a straight blade, a hooked blade, an injection needle, a grasping mechanism, and an energy probe.

18. The medical device of claim 13, further comprising:
an access tube configured to receive the guide therein.

19. The medical device of claim 13, wherein the guide is configured to receive electric energy.

20. The medical device of claim 13, further comprising:
at least one second tool having a distal end effector configured to perform a procedure.
